(19) United States
(12) Patent Application Publication
(10) Pub. No.: US 2014/0288461 A1
(43) Pub. Date: Sep. 25, 2014

(54) SWAB HAVING A LUER CONNECTION

(71) Applicant: FRIZ BIOCHEM GESELLSCHAFT FUR BIOANALYTIK MBH, Neuried (DE)

(72) Inventor: Gerhard Hartwich, Munchen (DE)

(73) Assignee: FRIZ BIOCHEM GESELLSCHAFT FUR BIOANALYTIK MBH, Neuried (DE)

(21) Appl. No.: 14/358,478

(22) PCT Filed: Sep. 21, 2012
(86) PCT No.: PCT/DE2012/100296

§ 371 Date: May 15, 2014

(30) Foreign Application Priority Data
Nov. 15, 2011 (DE) 20 2011 051 977.5

(51) Int. Cl. A61F 13/38; A61B 10/00; A61B 10/02

(52) U.S. Cl. CPC A61F 13/38; A61B 10/02; A61B 10/0096
USPC 600/572

(57) ABSTRACT

The invention relates to a swab system, comprising a sample tube, which is closed at the front end and open at the rear end, and a swab. The swab has an elongated, tubular swab rod, wherein the swab rod is provided with a sample-gathering means at the front end of the swab rod and a gripping piece at the rear end of the swab rod. The gripping piece closes the rear, open end of the sample tube in an accurately fitting manner and is equipped with a closing cap for covering the gripping piece in a sealed manner. The gripping piece has the internal taper of a Luer connection, wherein the internal taper of the Luer connection has a fluid connection to the tubular swab rod.

### FIELD OF THE INVENTION

The present invention relates to a swab having a Luer connection.

### BACKGROUND OF THE INVENTION

Conventional smear sampling swabs consist of a rod, made for example from wood, plastic or aluminium, which is provided at one of its ends with a sample-gathering means, e.g. a type of cotton pad, sponge or brush. For protection, the rod with the sample-gathering means is housed in a sample tube, wherein the rod, with its rear end remote from the sample-gathering means, is configured at the same time as a closure for the open side of the sample tube. Such swabs are used for example for streaking of fluids, such as bacterial suspensions, for applying ointments onto local body parts or for smears of body cavities.

The obtained smear samples must be protected as completely as possible from contamination. Thereby, receiving of the smear sample into a solvent and the transfer of the dissolved smear sample into a detection apparatus present great problems, which despite all precautionary measures frequently entail a contamination of the sample.

Therefore, a need still exists for swab systems which enable transportation and transfer of the smear sample into an examination apparatus in a manner as contamination-free as possible.

### DESCRIPTION OF THE INVENTION

It is therefore an object of the present invention to provide a swab system which ensures transport and transfer of the smear sample into an examination apparatus in a manner as contamination-free as possible.

This object is solved according to the invention by the swab system according to independent claim 1. Further advantageous details, aspects and embodiments of the present invention are evident from the dependent claims, the description, the drawings and the example.

The present invention provides a swab system comprising a sample tube which is closed at the front end and open at the rear end, and a swab. The swab has an elongated, tubular swab rod, which is provided at its front end with a sample-gathering means and at its rear end with a gripping piece, wherein the gripping piece closes the rear, open end of the sample tube in an accurately fitting manner. The gripping piece is additionally equipped with a closing cap for covering the gripping piece in a sealed manner. According to the invention, the gripping piece has the internal taper of a Luer connection, wherein the internal taper of the Luer connection has a fluid connection to the tubular swab rod.

A Luer connection is a standardized connection system for tubing systems in the medical field. It is used amongst others in cannulas, syringes, catheters, three-way valves or infusion tubes. The sealing of the connection is achieved by a tapered configuration of the connection parts, the so-called Luer cone. Thereby, the internal taper of the one connection side is denoted as “female”, the external taper of the opposite side as “male”. To secure the connection, the Luer cone may provide a thread with a union nut. In this case, the system is denoted as a Luer lock. The connection closes and opens with a half rotation.

Through the internal taper of a Luer connection, provided in the gripping piece of the swab rod, it becomes possible to connect a conventional syringe with external taper of a Luer connection to the swab system according to the invention. Through the fluid connection of internal taper with the interior cavity of the swab rod, a solvent which is provided in the syringe can be injected through the Luer connection into the hollow body of the swab rod. By light pressure onto the piston of the syringe, the solvent moves through the cavity of the swab rod and then penetrates the sample-gathering means at the front end of the swab rod from the interior outwards. In this way, the sample which is present on the outer side of the sample-gathering means is stripped quickly and with a high yield from the sample-gathering means.

By repeatedly aspirating and injecting the solvent or the solvent already mixed with sample, respectively, a complete intake of the sample into the solvent is achieved. At the end of the stripping process, the sample suspended in the solvent is present in the syringe, which is equipped with the external taper of the Luer connection. The sample may then be transferred without any difficulty into any kind of examination apparatus, which in turn is equipped with the internal taper of a Luer connection.

In this way, both the suspending of the sample and also the sample transfer can be carried out without removing the swab from the sample tube. The complete transfer of the sample from the sample-gathering means into the solution and subsequently into the examination apparatus is therefore possible in a contamination-free manner, owing to the Luer connection.

According to a particularly preferred embodiment of the present invention, two internal tapers are provided in the gripping piece. In this case, the Luer connection is designed as a Luer three-way valve. The external taper of a Luer connection provided on an examination apparatus can be connected with the second internal taper. The syringe connected to the first internal taper acts in this case also as a pump, which ensures intake of the sample into the solvent and then, with corresponding position of the internal tapers of the gripping piece, transfers the suspended sample directly from the sample tube via the syringe into the examination apparatus.

Particularly preferably, the swab and the sample tube are sterile. In this embodiment, the field of application of the swab system is not restricted to specific DNA analyses, rather, a versatile use in the medical and diagnostic field is possible.

Particular advantages emerge in that that gripping piece is equipped with an internal taper for a Luer lock connection. The additional securing by the Luer lock connection prevents the Luer connection between syringe and gripping piece from becoming detached accidentally.

According to a preferred embodiment of the present invention, the sample tube narrows conically for volume reduction in the region of its front end. The necessary volume of solvent for stripping the sample from the sample-gathering means can be reduced in this way, as the fluid level is sufficient, even with small quantities of fluid, in order to cover the sample-gathering means completely with solvent.

Equally preferably, the sample tube additionally can be equipped in the region of its front end with an insertion sleeve to receive the sample-gathering means. In this way also a volume reduction is possible for the volume of solvent necessary for dissolving the sample. For example, the insertion sleeve can be designed in the form of a sample vessel which is open on an upper side, wherein the insertion sleeve particularly preferably has a collar which is connected with the inner side of the wall of the sample tube. Through this funnel-like construction of the insertion sleeve it is ensured that excess solvent, which is pressed out from the syringe into the tubular swab rod and from there via the sample-gathering means into the insertion sleeve, does not arrive into the volume of the sample tube situated beneath the insertion sleeve.

The present invention also comprises a kit for sampling and transfer of body smears comprising one of the swab systems described above and a syringe equipped with the external taper of a Luer connection.

### Manner of Executing the Invention

The invention will be explained below in greater detail by reference to exemplary embodiments in connection with the drawings.

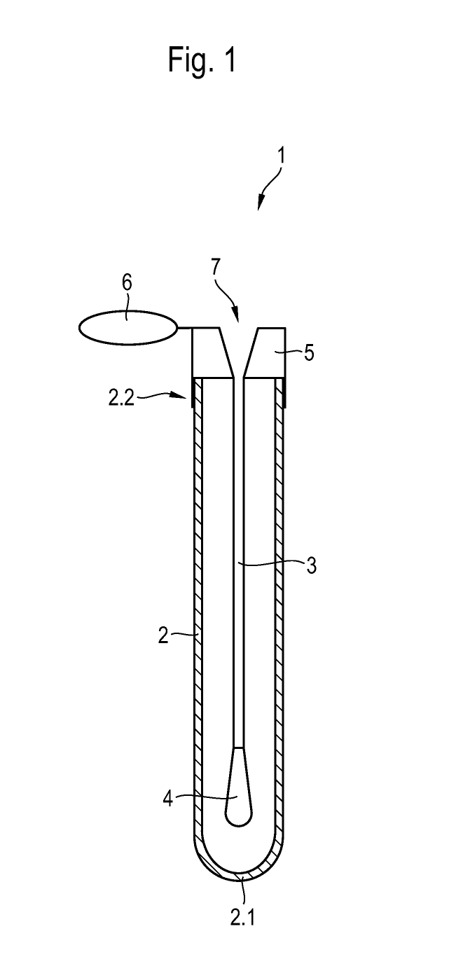

FIG. 1 shows in longitudinal section a swab system 1 according to the invention. The swab system 1 comprises a sample tube 2 which is closed at the front end 2.1 and open at the rear end 2.2, and a swab. The swab has a swab rod 3 designed as an elongated hollow body, wherein the swab rod 3 is provided at its front end with a pad of cotton wool as sample-gathering means 4 and at its rear end with a gripping piece 5. The gripping piece 5 encompasses the rear, open end 2.2 of the sample tube 2 and closes the latter in an accurately fitting manner.

The gripping piece 5 has the internal taper 7 of a Luer connection, wherein the internal taper 7 has a fluid connection with the tubular swab rod 3. The gripping piece 5 is equipped in addition with a closing cap 6 for covering the gripping piece 5 in a sealed manner. The closing cap 6 is closed for the contamination-free transport of the sample.

After the closing cap 6 is opened, the internal taper 7 of the Luer connection, provided in the gripping piece 5 of the swab rod 3, can be connected with the external taper provided on a conventional syringe with a Luer connection. Through the fluid connection of internal taper 7 with the interior cavity of the swab rod 3, a solvent provided in the syringe can be injected through the Luer connection into the hollow body of the swab rod 3. The solvent penetrates the sample-gathering means 4 from the interior outwards and in this way strips the sample present on the outer side of the sample-gathering means 4 quickly and with a high yield from the sample-gathering means 4.

By repeatedly aspirating and injecting the solvent or the solvent with suspended sample, respectively, a complete intake of the sample into the solvent is achieved. At the end of the stripping process, the suspended sample is present in the syringe, which is equipped with the external taper of the Luer connection. The sample can then be transferred without any difficulty into any kind of examination apparatus, which in turn is equipped with the internal taper of a Luer connection.

The complete transfer of the sample from the sample-gathering means 4 into the solution and subsequently into the examination apparatus is possible in a contamination-free manner owing to the Luer connection.

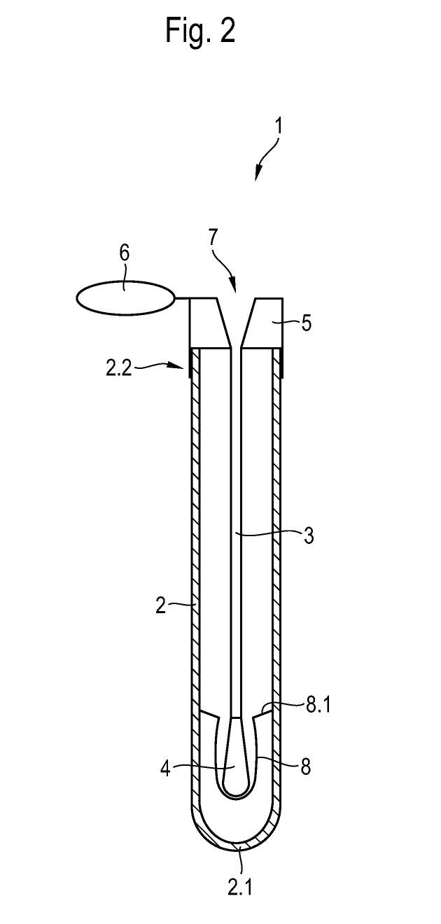

FIG. 2 shows a further embodiment of the swab system 1 according to the invention, in longitudinal section. The sample tube 2 of the illustrated swab system 1 is additionally equipped with an insertion sleeve 8 to receive the sample-gathering means 4. The insertion sleeve 8 is designed here with regard to its size and its dimensions such that the sample-gathering means 4 can in fact be completely received by the insertion sleeve 8, but when the sample-gathering means 4 is inserted, only a small free volume remains in the region of the sample-gathering means 4.

The insertion sleeve 8 is designed for example in the form of a sample vessel, open on an upper side, with a collar 8.1, wherein the insertion sleeve 8 is connected via the collar 8.1 with the inner side of the wall of the sample tube 2. For example, such a sample tube 2 with integrated insertion sleeve 8 can be produced by means of an injection moulding method.

Through the funnel-like construction of the insertion sleeve 8 illustrated in FIG. 2, it is ensured that solvent, which is pressed from the syringe into the hollow body of the swab rod 3 and from there via the sample-gathering means 4 into the insertion sleeve 8, does not arrive into the volume of the sample tube 2 situated beneath the insertion sleeve 8.

### Taking Samples if MRSA is Suspected

The practical application of the swab system 1 according to the invention is explained below by means of sampling if MRSA is suspected.

Bacteria of the genus Staphylococcus aureus may occur in humans and animals as a component of the skin flora. In humans, principally the front area of the nose and the inguinal region are colonized. MRSA (Methicillin-Resistant Staphylococcus Aureus) is a multi-resistant strain of Staphylococcus aureus and possesses the mecA resistance gene, which encodes a modified penicillin-binding protein (BPB2a)—transpeptidase. However, this modified penicillin-binding protein does not incorporate any beta-lactam antibiotics into the cell wall and leads to MRSA being resistant to all beta-lactam antibiotics (penicillins, cephalosporins, carbapenems). These resistant strains of bacteria in fact do not lead more frequently to infections than their antibiotic-sensitive relatives, however, an infection with an MRSA is substantially more difficult to treat. Therefore, attempts must be made to prevent the spread of MRSA, particularly in hospitals.

To take a sample from a patient with suspected MRSA, the swab rod 3 is removed from the sample tube 2 and—without contaminating the cotton wool pad 4 on the skin—is introduced as deeply as possible into the right Cavum nasi, is withdrawn thereafter and subsequently the same swab rod 3 is introduced into the left Cavum nasi, again as deeply as possible. On withdrawal of the swab rod 3, contact with the external skin is again to be avoided. The swab rod 3 is introduced back into the sample tube 2.

The microorganisms potentially adhering to the cotton wool pad 4 are
i. on the one hand to be kept alive as long as possible for a microbiological examination, but on the other hand are to be prevented from intensive growth and/or
ii. to be prepared for a PCR examination.

Therefore, a transport medium known to one skilled in the art, which fulfils the requirements with regard to (i) and/or (ii), is introduced into the swab system 1 via a commercially available syringe, pre-filled accordingly if applicable, equipped with the external taper of a Luer connection. Firstly, the closing cap 6 is opened, then the external taper of the syringe is placed onto the internal taper 7 of the gripping piece 5 of the swab rod 3 and the transport medium is pumped into the sample tube 2 via the internal taper 7 and the cotton wool pad 4 of the swab rod 3, and is subsequently sucked up again. This process is repeated several times. At the end of the procedure, the transport medium can be situated in the sample tube 2 and/or in the syringe.

The syringe and the swab rod 3 in the sample tube 2 can remain connected or can be separated from one another and closed respectively. In the case of the separated further use, the swab rod 3 can be used for example for a culture method, for which a nutrient medium is treated with the cotton wool pad 4 of the swab rod 3 (which is moistened with transport medium). The partially filled syringe can be used in order to carry out a PCR examination, wherein the transfer of the microorganisms (suspended/dissolved in the transport medium) ideally takes place via a renewed Luer connection into the device for further analysis by means of PCR.

### LIST OF REFERENCE SIGNS

1 swab system
2 sample tube
2.1 front end of the sample tube
2.2 rear end of the sample tube
3 swab rod
4 sample-gathering means
5 gripping piece
6 closing cap
7 internal taper of the Luer connection
8 insertion sleeve
8.1 collar

1. A swab system (1) comprising:
a sample tube (2), which is closed at the front end (2.1) and open at the rear end (2.2), and
a swab, wherein the swab has an elongated, tubular swab rod (3), wherein the swab rod (3) is provided at its front end with a sample-gathering means (4) and at its rear end with a gripping piece (5), wherein the gripping piece (5) closes the rear, open end (2.2) of the sample tube (2) in an accurately fitting manner, and the gripping piece (5) is equipped with a closing cap (6) for covering the gripping piece (5) in a sealed manner, characterized in that the gripping piece (5) has the internal taper (7) of a Luer connection, wherein the internal taper (7) of the Luer connection has a fluid connection with the tubular swab rod (3).

2. The swab system (1) according to claim 1, characterized in that two internal tapers (7) are provided, and the Luer connection is designed as a Luer three-way valve.

3. The swab system (1) according to claim 1, characterized in that the swab and the sample tube (2) are sterile.

4. The swab system (1) according to claim 3, characterized in that the gripping piece is equipped with an internal taper (7) for a Luer lock connection.

5. The swab system (1) according to claim 4, characterized in that the sample tube (2) narrows conically for volume reduction in the region of its front end (2.1).

6. The swab system (1) according to claim 5, characterized in that the sample tube (2) is additionally equipped in the region of its front end (2.1) with an insertion sleeve (8) to receive the sample-gathering means (4).

7. A kit for sampling and transfer of body smears, comprising a swab system (1) according to claim 1 and a syringe equipped with the external taper of a Luer connection.

8. The swab system (1) according to claim 2, characterized in that the swab and the sample tube (2) are sterile.

9. The swab system (1) according to claim 1, characterized in that the gripping piece is equipped with an internal taper (7) for a Luer lock connection.

10. The swab system (1) according to claim 2, characterized in that the gripping piece is equipped with an internal taper (7) for a Luer lock connection.

11. The swab system (1) according to claim 8, characterized in that the gripping piece is equipped with an internal taper (7) for a Luer lock connection.

12. The swab system (1) according to claim 1, characterized in that the sample tube (2) narrows conically for volume reduction in the region of its front end (2.1).

13. The swab system (1) according to claim 2, characterized in that the sample tube (2) narrows conically for volume reduction in the region of its front end (2.1).

14. The swab system (1) according to claim 3, characterized in that the sample tube (2) narrows conically for volume reduction in the region of its front end (2.1).

15. The swab system (1) according to claim 8, characterized in that the sample tube (2) narrows conically for volume reduction in the region of its front end (2.1).

16. The swab system (1) according to claim 11, characterized in that the sample tube (2) narrows conically for volume reduction in the region of its front end (2.1).

17. The swab system (1) according to claim 1, characterized in that the sample tube (2) is additionally equipped in the region of its front end (2.1) with an insertion sleeve (8) to receive the sample-gathering means (4).

18. The swab system (1) according to claim 2, characterized in that the sample tube (2) is additionally equipped in the region of its front end (2.1) with an insertion sleeve (8) to receive the sample-gathering means (4).

19. The swab system (1) according to claim 3, characterized in that the sample tube (2) is additionally equipped in the region of its front end (2.1) with an insertion sleeve (8) to receive the sample-gathering means (4).

20. The swab system (1) according to claim 4, characterized in that the sample tube (2) is additionally equipped in the region of its front end (2.1) with an insertion sleeve (8) to receive the sample-gathering means (4).
